(19) United States
(12) Patent Application Publication
(10) Pub. No.: US 2005/0222081 A1
(43) Pub. Date: Oct. 6, 2005

(54) Cross-linked hyaluronate compounds

(76) Inventors: Diego A. Gianolio, Boston, MA (US); Pericles Calias, Melrose, MA (US); Robert J. Miller, E. Bridgewater, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 10/511,373

(22) PCT Filed: Apr. 17, 2003
(86) PCT No.: PCT/US03/11830

§ 371 Date: May 4, 2005

(30) Foreign Application Priority Data
Apr. 17, 2002 (US) 60373279

(51) Int. Cl. A61K 31/728; C08B 37/00; C07D 203/04

(52) U.S. Cl. 514/54; 536/53; 548/954

(57) ABSTRACT

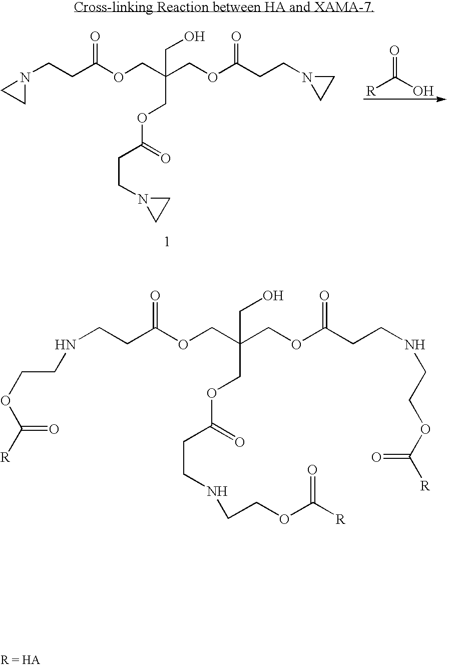

There is disclosed a cross-linked hyaluronate compound obtained by reacting a sodium hyaluronate compound with a polfunctional aziridine compound as a cross-linking agent. The polyfunctional aziridine compound was added to hyaluronan of various molecular weights in an equivalent ratio of HA/AZ of 1:1 to 1:10, preferably, 1:3 to 1:10, more preferably, 1:3 to 1:5; most preferably 1:4 to 1:5. The polymeric hydrogel compounds obtained are useful to prevent the formation of post-operative adhesions, designing tissue engineering applications, and the like.

### BACKGROUND OF THE INVENTION

1. Field of the Invention

This invention is directed to cross-linked hyaluronate polymeric compounds containing hydrolizable linkages that are capable of biodegradation. Even more particularly, this invention is directed toward the use of such cross-linked hyaluronate polymers as products for post-surgical adhesion prevention and/or drug delivery and as viscoelastic supplements.

2. Description of the Related Art

The synthesis of aziridine, diaziridine and polyfunctional aziridine compounds are known and various uses of such compounds are also known.

For example, European Patent Application 584,629 to Bender, et al. discloses the use of diaziridine compounds to increase the viscosity of lubricating oils. G. Sosnovsky, et al. (J. Cancer Res. Clin. Oncol., 107(3), 217-220 [1989]) discloses the use of diaziridine compounds as anti-tumor agents. French Patent 1,534,452 to Burns, et al. discloses the use of diaziridine compounds as an adhesive for rubber compounds. U.S. Pat. No. 3,376,263 to Ishida discloses the use of diaziridine compounds as a catalyst to prepare high molecular weight poly(oxymethylene).

It is also well known to use aziridine compounds in reactions with various functional groups.

For example, S. Nishimoto, et al. (J. Polym. Sci., Polym. Lett. Ed., 22(6), 323-326 [1984]) discloses the reaction of aziridine compounds with hydroxyls. U.S. Pat. No. 3,828,024 to Breslow discloses the reaction of aziridine compounds with groups containing double bonds. U.S. Pat. No. 3,468,818 to Phillips discloses the reaction of aziridine compounds with carboxyl groups. W. M Coull, et al. (Synthesis, 10, 1347 [2000]) discloses the reaction of aziridine compounds with nucleophiles.

In addition, it is known to use triaziridine compounds to impart fire retardency to materials.

For example, W. Tsuji, et al. (Bull. Inst. Chem. Res., 50(2), 83-93 [1972]) discloses the reaction of triaziridine compounds with acrylic acid to obtain a material that can be grafted onto cotton to increase its fire retardency. T. Ikeda, et al. (Sim'l Gakkaishi, 30(5-6) T292-T298 [1974]) discloses the reaction of triaziridine compounds with acrylic acid to obtain a material that can be grafted onto polypropylene to increase its fire retardency.

Sodium hyaluronate (HA) is a linear polysaccharide having alternating Beta-1-3-D-glucuronic acid and β-1-4-N-acetyl-D-glucosamine units and is one of the components of the extracellular matrix, the synovial fluid of joints, and the scaffolding comprising cartilage. [N. Larson, et al., Mater, Res. Soc. Symp. Proc., 394 (149-153); T. Pouyani, et al., Bioconjugate Chem., 1994, 5 (339-347); T. C. Laurent, et al., B. Acta Chem., Scand., 1984, 18 (274-275); Y. Nobuhiko, et al., S. J. Controlled Release, 1993, 25 (1-2) (133-143); Y. Nobuhiko, et al., S. J. Controlled Release, 1992, 22(2) (105-116)] The molecular weight of this material is generally in the range from about 50 kDa to about 8×10³ kDA depending upon the supply source, the method of isolation and the method of determination. Sodium hyaluronate may be derived from animal and bacterial sources.

HA and commercially produced cross-linked derivatives of HA exhibit remarkable viscoelastic properties [A. Atti, et al., Tissue Cell, 2001, 33(3) (294-300); G. Herrero-Beaumont, et al., Clin. Chem Acta, 2001, 308(1-2) (107-115)] and account for their usefulness in joint lubrication. The immunoneutrality of HA provides an excellent building block for the development of novel biocompatible and biodegradable biomaterials. [D. Pressato, et al., Pct Int. Appl., 1997 Fidia Adv. Biopol, S.R.L., [Italy IT 95-166 19950829].

Most of the cross-linked forms of HA are hydrogels that exhibit different handling properties depending upon their cross-link densities. It would be desirable to obtain HA-based hydrogels that can be used in medical application such as preventing the formation of post-operative adhesions, designing tissue engineering applications, and the like. However, it is known that irreversible cross-linking of HA retards dissolution and resorption of HA hydrogels resulting in unacceptably long residence times in the body.

Among the cross-linking agents that have been used to obtain HA hydrogels are 1,4-butanediol diglycidyl ether and divinyl sulfone (DVS). Both of these molecules react with the hydroxyl groups of HA forming intermolecular ether bonds that are stable under physiological conditions. However, the dissolution and resorption rates of the resulting cross-linked HA is very slow leading to unacceptably long residence times.

It is also known that an aziridine can react with the carboxyl group of HA to form an ester bond. For example, GB 2,151,244A of Balazs et al. discloses the preparation of water insoluble, biocompatible HA of sufficiently long in vivo residence time to serve as artificial heart valves and vascular grafts. Balazs specifically teaches subjecting hyaluronic acid to treatment with a cross-linking agent such as a polyaziridine at molar ratios of hyaluronic acid to cross-linking agent of at least 2 to 1.

These prior art references do not disclose or suggest that aziridine compounds can be used to obtain cross-linked hyaluronate compounds having a desired or pre-determined and acceptably short in vivo residence times.

### SUMMARY OF THE INVENTION

It has now been found that novel polymeric, cross-linked hyaluronate compounds containing hydrolizable linkages can be obtained and utilized as a surgical product such as, for example, preventing the formation of post-operative adhesions, engineering tissue development, and the like.

In one embodiment of this invention, cross-linked hyaluronan having hydrolizable linkages (i.e., esters) are synthetically prepared.

In another embodiment, a polyfunctional aziridine compound is used as a cross-linking agent to obtain cross-linked hyaluronan hydrogels having different molecular weights.

In another embodiment, a polyfunctional aziridine compound is used as a cross-linking agent to obtain cross-linked hyaluronan compound having a range of equivalent ratio of hyaluronan to aziridine of 1:1 to 1:10. The molecular weight of the hyaluronan used may be 500 kDaltons or more. The polyfunctional cross-linking agent include, but are not limited to, di- and tris-aziridine cross-linking agents such, as 1,1′,1″-methylidynetris-aziridine; 1,1′,1Δ-methylidynetris[2,2-dimethyl]-aziridine; 1,1′-[2-(1-aziridinylmethyl)-1,3-propanediyl]bis-aziridine; 1-aziridinepropanoic acid, 2,2-bis[[3-(1-aziridinyl)-1-oxopropoxy]methyl]-1,3-propanediyl ester; 1-aziridinepropanoic acid, 2-propyl-, 2-(hydroxymethyl)-2-[[1-oxo-3-(2-propyl-1-aziridinyl)propoxylmethyl]-1,3-propanediyl ester; 1-aziridinepropanoic acid, 2,2-dimethyl-, 2-[[3-(2,2-dimethyl-1-aziridinyl)-1-oxopropoxy]ethyl]-2-(hydroxymethyl)-1,3-propanediyl ester; di[2-(1-aziridinyl)ethyl)]adipate; 1,3-bis(1-aziridinyl)-3-phenyl-1-propanol; 1,1′-(1,3-propanediyl)bis-2-aziridinecarbonitrile; α,β-bis(1-aziridinyl) 2-furanpropanol; 1-[3-(1-aziridinyl)propionyl]-aziridine; 1,3-bis(1-aziridinyl)-2-propanol; 1,3-bis(2-methyl-1-aziridinyl-)-2-propanol; (1-aziridinylpyruvoyl)-,1-[(p-nitrophenyl)hydrazone]aziridine; and 1,1′-(1,3-dioxo-1,3-propanediyl)bis-aziridine, pentaerythritol tris(3-aziridinopropionate) and trimethylolpropane tris[3-(2-methylaziridinyl)propanoate].

A process of making a compound of the present invention, comprising hyaluronan cross-linked with a polyfunctional cross-linking agent having two or more aziridines, includes the steps of providing a hyaluronan solution at a pH of 4 to 10, and reacting the hyaluronan with the polyfunctional cross-linking agent. This reaction may be done by reacting hyaluronan with the polyfunctional cross-linking agent at an equivalent ratio of hyaluronan to aziridine of 1:1 to 1:10, preferably, 1:3 to 1:10, more preferably, 1:3 to 1:5; most preferably 1:4 to 1:5. The polyfunctional cross-linking agent may have two aziridines, and preferably three aziridines. The process may use hyaluronan having a molecular weight of 500 kDaltons or more to cross-link with the polyfunctional cross-linking agent. Selection of reactants and process conditions leads to compounds having different viscosity, phase angle and complex modulus, as well as different rates of biodegradation. The preferred physical characteristics of the compound are dictated by its intended application, a few of which are outlined below.

Cross-linked hyaluronan can be of different physiological characteristics, ranging from flowing to rubber-like ones. For example, a hydrogel having non-flowing characteristics would be preferable for surgical applications such as a surgical drug delivery vehicle (to form drug depot implanted through a surgical incision or as an auxiliary procedure during or following a surgical procedure). A hydrogel having flowing characteristics would be preferable for viscosupplementation for ophthalmologic application and articular application.

The compounds of the present invention may be combined with a pharmacologically active agent to produce a pharmaceutical composition. The compounds of the present invention may also be used to prevent post-operative surgical adhesions of tissue by providing the tissue surfaces involved in the surgery with a hydrolyzable coating comprising these compounds. The coating to prevent such post-operative surgical adhesions may be in the form of a gel, membrane, foam, or fiber. The coating may also contain a pharmacologically active agent.

The compounds of the present invention may also be used for viscosupplementation in medical applications which comprises contacting body tissue with a biocompatible viscoelastic gel slurry comprising the compounds of the present invention. The gel slurry may optionally contain a pharmacologically active agent.

### BRIEF DESCRIPTION OF THE DRAWINGS

FIG. 1 shows a cross-linking reaction between hyaluronan (HA) and pentaerythritol tris(3-aziridinopropionate) (XAMA-7, 1).

FIG. 2 shows the trend of viscosity as a function of pH. The most viscous gel was obtained at a pH=9.0.

FIG. 3 shows the trend of viscosity, phase angle and complex modulus as a function of equivalents of HA per equivalents of aziridine (AZ) from XAMA-7 added.

FIG. 4A-C show the trend of viscosity, phase angle and complex modulus as a function of time for 1:1, 1:2, and 1:3 equivalent ratios of HA:AZ from XAMA-7.

FIG. 5 shows the trend of viscosity, and phase angle as a function of equivalents of HA per equivalents of AZ from XAMA-7 that is added, for more and less homogeneous reaction mixtures.

FIGS. 6A-C show the trend of viscosity, phase angle and complex modulus of the gel in PBS at 37° C. as a function of time. Data points marked with ♦ relates to crosslinked gels with equivalent ratio of HA:AZ of 1:4. Data points marked with ▪ relates to HA-DVS gels.

### DETAILED DESCRIPTION OF THE PRESENTLY PREFERRED EMBODIMENTS

We have developed a synthetic approach to prepare cross-linked HA with hydrolizable linkages (i.e., esters) having higher density of cross-links than have been previously reported. A polyfunctional aziridine cross-linking reagent was added to hyaluronan of various molecular weights.

Hyaluronan is a polysaccharide consisting of repeating units of glucuronic acid and N-acetylglucoseamine (HA). It has a molecular weight ranging from about 0.5×10⁵ to about 8×10⁶ Daltons, depending on the source of its extraction. Hyaluronan is naturally expressed in developing and healing tissues and has the capacity to bind large amounts of water. Hyaluronan (HA) as used herein include hyaluronic acid and salts thereof.

The hyaluronan is crosslinked with a polyfunctional aziridine cross-linking reagent in a range of equivalent ratios of hyaluronan to aziridine. As used herein, the equivalent ratio is a ratio of the number of repeating glucuronic acid-glucosamine units in the hyaluronan (each unit having a molecular weight of about 401) to the number of aziridine rings provided by the polyfunctional aziridine cross-linking reagent. For hyaluronan cross-linked with an aziridine crosslinking agent, it is preferred that the equivalent ratio of hyaluronan to aziridine (HA:AZ) is 1:1 to 1:10, preferably, 1:3 to 1:10, more preferably, 1:3 to 1:5; most preferably 1:4 to 1:5. Cross-linked polymers were quickly obtained, characterized, and their physical properties were studied. The rate of gel breakdown was measured and gel breakdown occurred easily under physiological conditions, releasing benign side products.

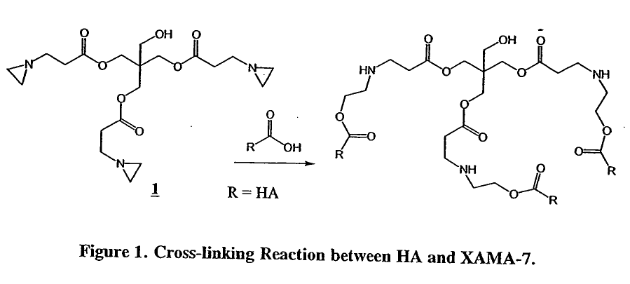

The polyfunctional aziridine cross-linking agent may be selected from a large group of compounds. One particulary preferred reagent is pentaerythritol tris(3-aziridinopropionate), (XAMA-7, 1). This reagent can react with the carboxyl group of HA to form an ester bond without releasing secondary products (see FIG. 1). Ester bonds undergo hydrolysis more easily under physiological conditions, therefore HA containing polyesters in the crosslinks should dissolve and clear from the body faster leading to a shorter residence time. Our work describes the synthesis, characterization, and degradation studies of HA crosslinked with XAMA-7.

While XAMA-7 is the preferable polyfunctional cross-linking agent to react with hyaluronan, other polyfunctional cross-linking agents having two or more aziridines may also be used. These agents include, but are not limited to, diaziridines such as di[2-(1-aziridinyl)ethyl)]adipate, pentaerythritol tris(3-aziridinopropionate), discussed in GB 2,151,244 and 2,151,246 of Balazs et al.; 1,3-bis(1-aziridinyl)-3-phenyl-1-propanol; 1,1′-(1,3-propanediyl)bis-2-aziridinecarbonitrile, discussed in German Patent DE 2163623; α,β-bis(1-aziridinyl) 2-furanpropanol; 1-[3-(1-aziridinyl)propionyl]-aziridine; 1,3-bis(1-aziridinyl)-2-propanol; 1,3-bis(2-methyl-1-aziridinyl)-2-propanol; (1-aziridinylpyruvoyl)-,1-[(p-nitrophenyl)hydrazone]aziridine; 1,1′-(1,3-dioxo-1,3-propanediyl)bis-aziridine, and those diaziridines disclosed in Andersson et al., Tetrahedron 54(38), 11549 (1998); Tanner et al., Acta Chem. Scand. 50(4), 361 (1996); Olivier et al., J. Org. Chem. 60(15), 4884 (1995); Russian Patent SU 1723125 (bisaziridine alkanes); Kadorkina et al., Izv. Akad. Nauk SSSR, Ser. Khim. 4, 882 (1991); Manecke et al., Makromol. Chem. 175(6), 1833 (1974); Manecke et al. German Patent DE 1270287; Watanabe et al., Kogyo Kagaku Zasshi 72(6), 1349 (1969); and Hillers et al.; Bestian et al., German Patent DE 1243687.

The compositions of the invention may further include a drug or pharmacologically active agent for use as a drug delivery system. The particular drug used is a matter of choice depending on the intended use of the composition. Preferred drugs include, but are not limited to, proteins (e.g., growth factors, enzymes), steroids, non-steroidal anti-inflammatory drugs, cytotoxic agents (e.g., anti-tumor drugs), antineoplastics, antibiotics, antivirals, oligonucleotides (e.g., antisense), and biopolymers. When provided for cell and tissue growth and proliferation, the compositions of the invention may further include growth factors, and cell attachment proteins or peptides, as well as. When provided for the prevention of adhesions after surgery, the compositions of the invention may be used alone or may further include a drug or pharmaceutical agent such as an anti-inflammatory drug.

The compositions of the invention may be formed into films, foams, or gels for drug delivery. For example, in the case where rapid, localized delivery is desirable, rapidly degradable compositions within the invention can be used. Alternatively, compositions that degrade at a slower rate are useful for sustained release drug delivery. The drug to be delivered can be dispersed within the composition, or can be covalently bonded to the foam, film, or gel as described, for example, in R. V. Sparer et al., 1983, Chapter 6, pages 107-119, in T. J. Roseman et al., Controlled Release Delivery Systems, Marcel Dekker, Inc., New York; and the foam, film, or gel can then be implanted or injected at the locus where delivery is desired.

The compositions of the invention may be used to repair articular cartilage defects in a mammal. The surfaces of articulating bones in mammalian joints are covered with articular cartilage. These joints include those located in the knee, hip and shoulder. The articular cartilage prevents direct contact of the opposing bone surfaces and permits the near frictionless movement of the articulating bones relative to one another. Defects in the articular cartilage may result from degenerative joint diseases, for example, during osteoarthritis. Repair of such articular cartilage defects may be accomplished by implanting into the cartilage defects the compositions of the present invention.

Hyaluronan solutions have been used clinically in ophthalmologic surgery due to the unique viscoelastic properties of the material. Because of the high viscosity, administered hyaluronan solutions are retained in the anterior chamber of the eye and serve to protect fragile corneal endothelial surfaces during intraocular lens implantation (Pape et al., Ophthalmology 87:699, 1980). The compositions of the present invention may be advantageously used in such ophthalmologic applications.

The compositions of the present invention may also be advantageously used in tissue engineering applications, including but are not limited to, as a support for cardiac tissue, bladder tissue, nerve tissue, kidney tissue, bone cells, intestinal tissue, and pancreatic tissue. Such a support would be impregnated with the desired tissue cells, placed in an area of the body requiring such tissue cells, and the support would degrade leaving the tissue cells implanted in this area of the body.

The compositions of the present invention may also be advantageously used to coat medical devices that come into contact with bodily fluids and tissue so as to prevent biofouling of the surfaces of the medical devices during use.

The above-described applications of the compositions of the invention require certain rheological properties and degradation rates. For example, where the compositions are used to prevent adhesions after surgery, the phase angles of the compositions to be used are preferably equal to or less than 50° and the complex moduli are equal to or greater than 30 Pas. Such an application typically requires that the compositions have a residence time of about 24 to 72 hours before complete degradation occurs. Similar Theological properties and degradation rates are preferred where the compositions are used for the delivery and implantation of cells into certain parts of the body. Where the compositions are used for drug delivery, the preferred rheological properties are also similar to those for adhesion prevention and cell implantation. The delivery of drugs, however, typically requires longer residence times of greater than about 24 hours to several weeks. Where the compositions are used for repairing articular cartilage defects, the preferred rheological properties are also similar to those for adhesion prevention and cell implantation. Typically, it is preferred in such an application that the phase angle be as low as possible so as to be as fluid as possible. The repair of articular cartilage defects typically requires residence times of about two weeks to several weeks.

Materials and Methods

Sodium hyaluronate (500, 1,500, 2,100 kDa) was prepared by bacterial fermentation.

High molecular weight (MW) HA (1.5 and 2.1×10³ kDa) was prepared from Streptococcus fermentation and low molecular weight (MW) HA was obtained by γ-irradiation of high MW HA using known methodology (K. Vercruysse, et al., Crit. Rev. Ther. Drug Carr. Syst., 15 (513-555); G. Prestwich, et al., The Chem Biol. And Med. Appl. of HA and its Deriv., 1998, Laurent Ed., Portland Press, London (43-65); L. Freed, et al., Biotechnology, 1994, 12 (689-693)].

Pentaerythritol tris(3-aziridinopropionate) (XAMA-7) was purchased from Sybron Chemicals Inc. NJ, and used without further purification. Viscosity (η), phase angle (δ) and complex modulus (G*) measurements were performed on a Bohlin Instruments (model INT CVO 50) rheometer. Gel degradation rates were measured at physiological pH at 37° C. Gel rheology was measured at the start of incubation and at various times out to 7 days. HA crosslinked with DVS having similar rheological properties was tested as a control. The effect of pH, HA MW, HA concentration, reagent stoichiometry and homogeneity of the reaction mixture on the resulting gel Theological properties and gel degradation rates were studied. For each set of conditions, three gel replicates were measured and averaged.

Experimental

Gel Formation

Several parameters influenced the degree of crosslinkage obtained when a freshly prepared 0.35 M solution of XAMA-7 in water was added to a solution of HA. We varied each one of these parameters until we determined the conditions that yielded the best results. For each set of conditions values of three replicates were measured and averaged. We tested these reactions on a 4 mL scale for each sample.

1) pH of the Reaction

HA solutions were allowed to cure in acidic conditions at varying pH by adding 0.1 N HCl prior to the addition of XAMA-7. Mild basic conditions (pH=9) were also tested by adding XAMA-7 to neutral solutions of HA.

### EXAMPLE I

The following protocol produces a crosslinked gel having an equivalent ratio of HA:AZ of 1:1. A 0.5% w/w solution of HA with a MW of 1.5×10⁶ Da was prepared. 4 mL of the HA solution (0.050 meq) was placed in a 50-mL round bottom flask, and the pH was adjusted to 3.5 with 0.1 N HCl. To this solution 50 μL (0.017 mmol of XAMA-7 or 0.05 meq of AZ) of a freshly prepared 0.35 M solution of XAMA-7 were added. The solution was stirred briskly for 5 minutes on a vortex and then allowed to cure at room temperature for 4 hours to produce a crosslinked gel having an equivalent ratio of HA:AZ of 1:1. Rheology values were measured and the gel obtained had a viscosity of 0.79 Pas.

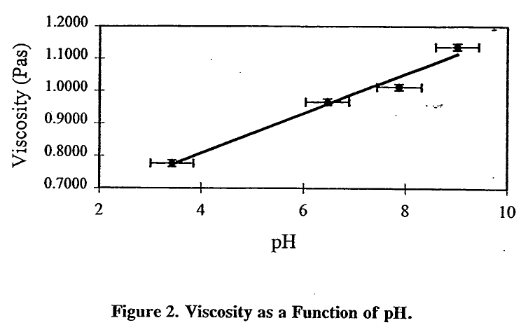

Additional crosslinked gels were made by adjusting the pH of the HA solution used to react with the XAMA-7 solution. The viscosity of the crosslinked gel was determined as a function of the pH of the HA solution in the range of 3.5 to 9 and summarized in FIG. 2.

2) MW and Concentration of HA

HA of three different MW: 500, 1.5×10³ and 2.1×10³ kDa was used. HA solutions were prepared with concentrations ranging from 0.5% to 3.0% w/w as follows.

### EXAMPLE II

A 1.0% w/w solution of HA with a MW of 1.5×10⁶ Da was prepared. 4 mL of the HA solution (0.10 meq) were placed in a 50 mL round bottom flask. To this solution 95 μL (0.03 mmol) of a freshly prepared 0.35 M solution of XAMA-7 were added for an equivalent ratio of HA:AZ of 1 to 1. The pH of the mixed solution was 9.0. The solution was stirred briskly for 5 minutes on a vortex and then allowed to cure at room temperature for 4 hours. Rheology values were measured and the gel obtained had a viscosity of 14.07 Pas.

Additional crosslinked gels were made by adjusting the MW of the HA used to react with the XAMA-7 solution. In some cases, the initial concentration of the HA solution was also adjusted. A summary of the viscosity values for products produced by varying the MW of HA and the initial concentration of HA is in Table 1.

Table 1 summarizes viscosity values measured for products obtained using varying HA MW and starting HA concentration.

TABLE 1
The Effect of HA Concentration and MW on Cross-
linked HA

| Concentration % w/w | MW (× 10⁶ Da) | η (Pas) |
|---|---|---|
| 0.5 | 1.5. | 1.14 |
| 1.0 | 1.5 | 14.07 |
| 1.0 | 2.1 | * |
| 1.5 | 1.5 | * |
| 3.0 | 0.5 | 1.09 |

Gel Viscosity (* unable to measure)

3) Amount of XAMA-7 Added

The amount of XAMA-7 added relative to HA ranged from 1:1 to 1:10 equivalents of HA to AZ in the following examples.

### EXAMPLE III

A 1.0% w/w solution of HA with a MW of 1.5×10⁶ Da was prepared. 4 mL of the HA solution (0.10 meq) were placed in a 50 mL round bottom flask. To this solution 0.5 mL (0.17 mmol) of a freshly prepared 0.35 M solution of XAMA-7 were added. For this example, 5 equivalents of AZ from XAMA-7 were added for each equivalent of HA (HA:AZ is 1:5). The pH of the mixed solution was 9.0. The solution was stirred briskly for 5 minutes on a vortex and then allowed to cure at room temperature for 4 hours. Rheology values were measured and the gel obtained had viscosity=110.00 Pas, phase angle=21.40°, and complex modulus=73.34 Pa.

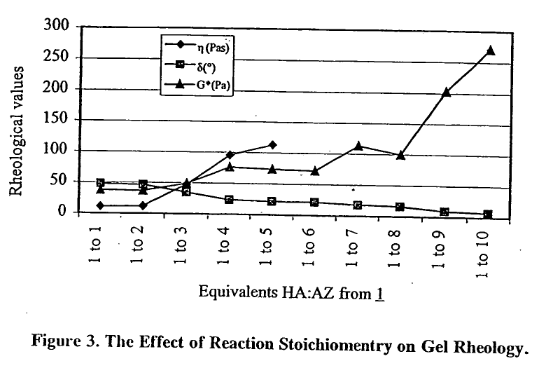

Additional crosslinked gels were made by adjusting the equivalent ratio HA:AZ. Rheological values of viscosity, phase angle and complex modulus for gels of varying equivalent ratios of HA/AZ are summarized in FIG. 3.

4) Time

Rheological values were measured at 4 and 21 hours after XAMA-7 was added to a HA solution.

### EXAMPLE IV

A 1.0% w/w solution of HA with a MW of 1.5×10⁶ Da was prepared. 4 mL of the HA solution (0.10 meq) were placed in a 50 mL round bottom flask. To this solution 0.3 mL (0.10 mmol) of a freshly prepared 0.35 M solution of XAMA-7 were added. For this example, 3 equivalents of AZ from XAMA-7 were added for each equivalent of HA (HA:AZ is 1:3). The pH of the mixed solution was 9.0. The solution was stirred briskly for 5 minutes on a vortex and then allowed to cure at room temperature for 21 hours. Rheology values were measured and the gel obtained had viscosity=92.26 Pas, phase angle=22.60°, and complex modulus=81.37 Pa.

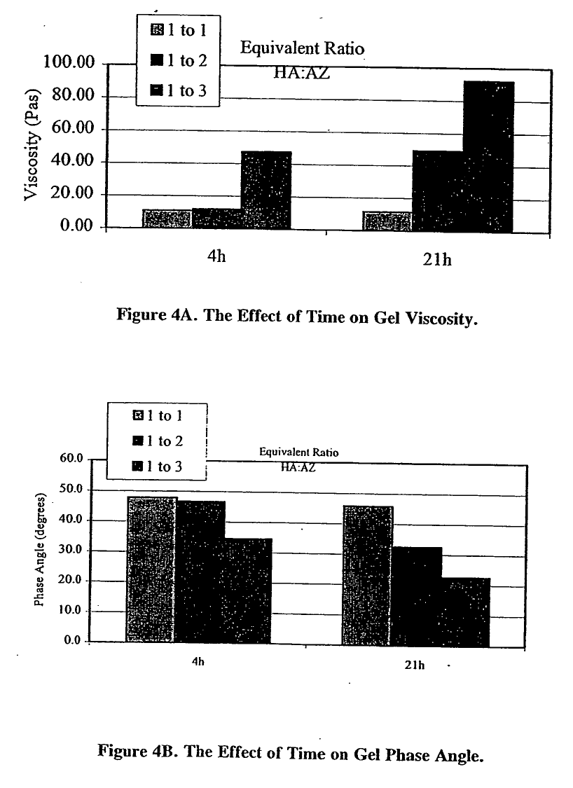

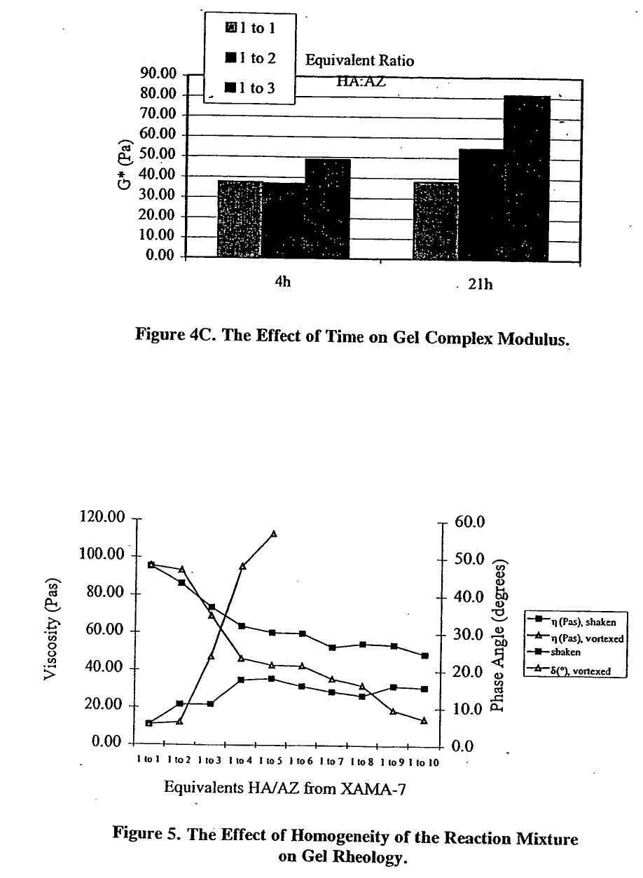

Additional crosslinked gels were made by adjusting the equivalent ratio HA:AZ and the time the gels were examined after the XAMA-7 was added to a HA solution. FIGS. 4A, B and C summarizes the change in viscosity, phase angle and complex modulus of HA/XAMA-7 compounds as a function of varying equivalent ratios and of time.

5) Homogeneity of the Reaction Mixture

Solutions were briefly shaken by hand or stirred briskly for 5 min on a vortex to reach a higher level of homogeneity, and then allowed to cure.

### EXAMPLE V

A 1.0% w/w solution of HA with a MW of 1.5×10⁶ Da was prepared. 4 mL of the HA solution (0.10 meq) were placed in a 50 mL round bottom flask. To this solution 0.5 mL (0.17 mmol) of a freshly prepared 0.35 M solution of XAMA-7 were added. For this example, 5 equivalents of AZ from XAMA-7 were added for each equivalent of HA (HA:AZ is 1:5). The pH of the mixed solution was 9.0. The solution was stirred briskly for 5 minutes on a vortex and then allowed to cure at room temperature for 4 hours. Rheology values were measured and the gel obtained had viscosity=112.84 Pas, and phase angle=21.4°.

Additional crosslinked gels were made by adjusting the equivalent ratio HA:AZ. The viscosity and phase angle of the HA/XAMA-7 compounds were determined at different equivalent ratios of HA/AZ, and are summarized in FIG. 5.

A. Gel Degradation

The gel used for the degradation study was obtained at pH=9.0, using 1.5×10³ kDa MW HA, 1.0% w/w, t=4 hours, and 1:4 equivalents of HA to AZ from XAMA-7. Rheological values were measured at t=0 and then at intervals during the next 7 days. HA-DVS with similar rheological properties was also tested as a control. We tested these reactions on a 40 mL scale for each sample.

Hydrolysis of HA-XAMA-7 leads to pentaerythritol and N-(2-hydroxymethyl)-β-alanine as side products. Both products are listed as non toxic in Material Safety Data Sheets

### EXAMPLE VI

36 mL of HA-XAMA-7 gel was placed in a 100 mL round bottom flask. The pH was adjusted to 7.0 with 1.0 N HCl, and 4 mL of 10× concentrated phosphate buffer saline solution (PBS) was added. The gel was mixed vigorously for 5 minutes until completely homogeneous. Rheology values were measured, viscosity=11.65 Pas, phase angle=40.08°, complex modulus=88.82 Pa. The gel was incubated at 37° C., and after 4 days the rheology values were measured again. Viscosity=5.07 Pas, phase angle=56.20°, complex modulus=94.62 Pa.

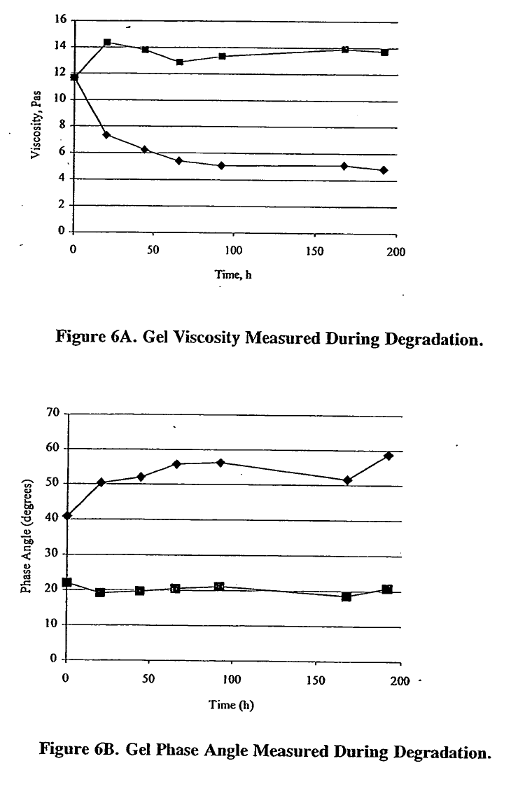

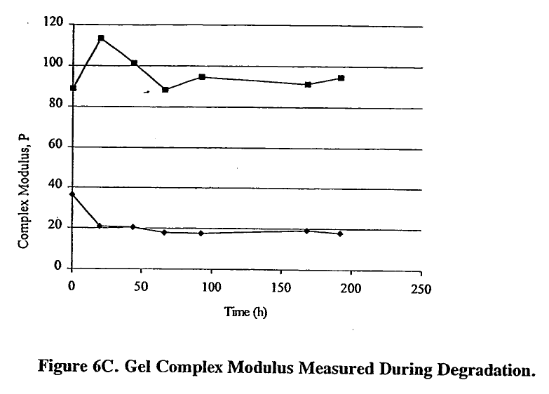

Changes in the viscosity, phase angle and complex modulus of the HA/XAMA-7 gel over time are summarized in FIGS. 6A, B and C.

HA-XAMA-7 appeared to decompose almost completely after a 2-day period. In contrast, HA-DVS maintained almost constant rheological values during a 7-day period.

A polyfunctional aziridine, XAMA-7, can be used as a crosslinking reagent for HA to form gels containing hydrolizable linkages. Products were obtained in a straightforward manner by adding a freshly prepared solution of XAMA-7 to a solution of HA. The degree of crosslinkage obtained depended on several parameters, and as a consequence it was possible to obtain gels of different handling properties, ranging from flowing to rubber-like ones. The degree of crosslinkage was determined measuring viscosity (η), phase angle (δ) and complex modulus (G*).

We found it more convenient to cure the solutions of HA at pH=9.0, using 1.5 kDa MW HA at a concentration of 1% w/w solutions. We let the reaction cure for 4 hours, using 1:4 equivalents of HA to AZ from XAMA-7. Better results were obtained when the reagents were mixed thoroughly. Gels obtained in these conditions proved to be stable in physiological conditions for a period of 2 days.

### EXAMPLE VII

### Synthesis of Di[2-(1-Aziridinyl)ethyl]Adipate

The titled diaziridine compound was synthesized following the procedure disclosed in U.S. Pat. No. 3,338,885 to Coker, et. al. The diaziridine compound may also be used to react with HA to form crosslinked HA gels.

Aziridineethanol and dimethyl adipate were obtained pure by distillation under high vacuum and kept under a nitrogen atmosphere. Into a dry 500 mL distillation flask there was placed 91 mL (1.14 mole) of aziridineethanol to which there was added 495 mg of NaH (60% dispersion in mineral oil). The mixture was stirred for 30 min and the flask was vented. Dimethyl adipate (48 mL, 0.29 mole) was added to the mixture and the flask was connected to a distillation column. The resulting mixture was then heated and methanol was removed at reduced pressure during a period of one hour. Excess aziridineethanol was then removed by heating the mixture to 70 C at a pressure of 0.15 mm Hg. The desired product was then distilled off from the mixture at 130 C at a pressure of 0.15 mm Hg to obtain 49 g (0.18 mole, 60% yield) of di[2-(1-aziridinyl)ethyl]adipate as a colorless oil having the following analysis:

¹H NMR (400 MHz, DMSO_{d6}) δ=1.10-1.11(m, 4H, CH₂). 1.51-1.55(m, 8H, CH₂ ), 2.28-2.35(m, 8H, CH₂), 4.09(t, J=4.0 Hz, 4H, CH₂) ppm.

³C NMR (100 MHz, DMSO_{d6}) δ=24.6; 27.0; 33.8; 59.7; 64.2; 173.3 ppm

IR: 3064.42; 2952.38; 1731.46; 1454.54; 1264.33; 1174.82 cm⁻¹

HA hydrogels obtained following the above-described methodology have the potential to be used as rapidly degrading products for adhesion prevention, prevention of biofouling on the surfaces of medical devices and tissue engineering applications.

The various features of novelty which characterize the invention are pointed out with particularity in the claims annexed to and forming a part of the disclosure. For a better understanding of the invention, its operating advantages, and specific objects attained by its use, reference should be made to the drawing and descriptive matter in which there are illustrated and described preferred embodiments of the invention.

The invention is not limited by the embodiments described above which are presented as examples only but can be modified in various ways within the scope of protection defined by the appended patent claims.

Thus, while there have shown and described and pointed out fundamental novel features of the invention as applied to a preferred embodiment thereof, it will be understood that various omissions and substitutions and changes in the form and details of the compounds illustrated may be made by those skilled in the art without departing from the spirit of the invention. It is the intention, therefore, to be limited only as indicated by the scope of the claims appended hereto.

All references cited herein are incorporated in their entirety by reference.

1. A compound comprising hyaluronan cross-linked with a polyfunctional cross-linking agent having two or more aziridines selected from the group consisting of 1,1′,1″-methylidynetris-aziridine; 1,1′,1″-methylidynetris[2,2-dimethyl]-aziridine; aziridinylmethyl)-1,3-propanediyl]bis-aziridine; 1-aziridinepropanoic acid, 2,2-bis[[3-(1-aziridinyl)-1-oxopropoxy]methyl]-1,3-propanediyl ester; 1-aziridinepropanoic acid, 2-propyl-, 2-(hydroxymethyl)-2-[[1-oxo-3-(2-propyl-1-aziridinyl)propoxy]methyl]-1,3-propanediyl ester; 1-aziridinepropanoic acid, 2,2-dimethyl-, 2-[[3-(2,2-dimethyl-1-aziridinyl)-1-oxopropoxy]ethyl]-2-(hydroxymethyl)-1,3-propanediyl ester; di[2-(1-aziridinyl)ethyl)]adipate; 1,3-bis(1-aziridinyl)-3-phenyl-1-propanol; 1,1′-(1,3-propanediyl)bis-2-aziridinecarbonitrile; α,β-bis(1-aziridinyl) 2-furanpropanol; 1-[3-(1-aziridinyl)propionyl]-aziridine; 1,3-bis(1-aziridinyl)-2-propanol; 1,3-bis(2-methyl-1-aziridinyl)-2-propanol; (1-aziridinylpyruvoyl)-,1-[(p-nitrophenyl)hydrazone)aziridine; and 1,1′-(1,3-dioxo-1,3-propanediyl)bis-aziridine, wherein the equivalent ratio of hyaluronan to aziridine is 1:1 to 1:10.

2. The compound of claim 1, wherein the equivalent ratio of hyaluronan to aziridine is 1:3 to 1:10.

3. The compound of claim 1, wherein the equivalent ratio of hyaluronan to aziridine is 1:3 to 1:5.

4. The compound of claim 1, wherein the equivalent ratio of hyaluronan to aziridine is 1:4 to 1:5.

5. The compound of claim 1 wherein the molecular weight of the hyaluronan is 500 kDaltons or more.

6. The compound of claim 1 wherein the polyfunctional cross-linking agent is di[2-(1-aziridinyl)ethyl)]adipate.

7. A process of making a compound comprising hyaluronan cross-linked with a polyfunctional cross-linking agent having two or more aziridines selected from the group consisting of 1,1′,1″-methylidynetrisaziridine; 1,1′,1″-methylidynetris[2,2-dimethyl]-aziridine; 1,1′-[2-(1-aziridinylmethyl)-1,3-propanediyl]bis-aziridine; 1-aziridinepropanoic acid, 2,2-bis[[3-(1-aziridinyl)-1-oxopropoxylmethyl)-1,3-propanediyl ester; 1-Aziridinepropanoic acid, 2-propyl-, 2-(hydroxymethyl)-2-[[1-oxo-3-(2-propyl-1-aziridinyl)propoxy]methyl]-1,3-propanediyl ester; 1-aziridinepropanoic acid, 2,2-dimethyl-, 2-[[3-(2,2-dimethyl-1-aziridinyl)-1-oxopropoxylethyl]-2-(hydroxymethyl)-1,3-propanediyl ester; di[2-(1-aziridinyl)ethyl)]adipate; 1,3-bis(1-aziridinyl)-3-phenyl-1-propanol; 1,1′-(1,3-propanediyl)bis-2-aziridinecarbonitrile; α,β-bis(1-aziridinyl) 2-furanpropanol; 1-[3-(1-aziridinyl)propionyl]-aziridine; 1,3-bis(1-aziridinyl)-2-propanol; 1,3-bis(2-methyl-1-aziridinyl)-2-propanol; (1-aziridinylpyruvoyl)-,1-[(p-nitrophenyl)hydrazone]aziridine; and 1,1′-(1,3-dioxo-1,3-propanediyl)bis-aziridine, comprising providing a hyaluronan solution at a pH of 4 to 10, and reacting the hyaluronan with the polyfunctional cross-linking agent.

8. A process of making a compound comprising hyaluronan cross-linked with a polyfunctional cross-linking agent having two or more aziridines selected from the group consisting of 1,1′,1″-methylidynetrisaziridine; 1,1′,1″-methylidynetris[2,2-dimethyl]-aziridine; 1,1′-[2-(1-aziridinylmethyl)-1,3-propanediyl]bis-aziridine; 1-aziridinepropanoic acid, 2,2-bis[[3-(1-aziridinyl)-1-oxopropoxy]methyl]-1,3-propanediyl ester; 1-Aziridinepropanoic acid, 2-propyl-, 2-(hydroxymethyl)-2-[[1-oxo-3-(2-propyl-1-aziridinyl)propoxy]methyl]-1,3-propanediyl ester; 1-aziridinepropanoic acid, 2,2-dimethyl-, 2-[[3-(2,2-dimethyl-1-aziridinyl)-1-oxopropoxy]ethyl]-2-(hydroxymethyl)-1,3-propanediyl ester; di[2-(1-aziridinyl)ethyl)]adipate; 1,3-bis(1-aziridinyl)-3-phenyl-1-propanol; 1,1′-(1,3-propanediyl)bis-2-aziridinecarbonitrile; α,β-bis(1-aziridinyl) 2-furanpropanol; 1-[3-(1-aziridinyl)propionyl]-aziridine; 1,3-bis(1-aziridinyl)-2-propanol; 1,3-bis(2-methyl-1-aziridinyl)-2-propanol; (1-aziridinylpyruvoyl)-,1-[(p-nitrophenyl)hydrazone)aziridine; and 1,1′-(1,3-dioxo-1,3-propanediyl)bis-aziridine, comprising reacting hyaluronan with the polyfunctional cross-linking agent at a equivalent ratio of hyaluronan to aziridine of 1:1 to 1:10.

9. The process of claim 8 further comprising a step of adding a pharmacolgically active agent to the hyaluronan before reacting with the polyfunctional cross-linking agent.

10. The process of claim 8 further comprising a step of adding a pharmacolgically active agent to the compound comprising hyaluronan cross-linked with a polyfunctional cross-linking agent.

11. The process of claim 8 wherein the polyfunctional cross-linking agent has two aziridines.

12. The process of claim 8 wherein the polyfunctional cross-linking agent has three aziridines.

13. The process of claim 11 wherein the polyfunctional cross-linking agent is di[2-(1-aziridinyl)ethyl)]adipate.

14. The process of claim 8 further comprising the step of selecting hyaluronan having a molecular weight of 500 kDaltons or more to cross-link with the polyfunctional cross-linking agent.

15. The compound produced by the process of claim 8.

16. The pharmaceutical composition comprising the compound of claim 1 and a pharmacologically active agent.

17. A method of preventing post-operative surgical adhesions of tissue comprising providing the tissue surfaces involved in said surgery with a hydrolyzable coating comprising a compound comprising hyaluronan cross-linked with a polyfunctional cross-linking agent having two or more aziridines, wherein the equivalent ratio of hyaluronan to aziridine is 1:1 to 1:10.

18. The method of claim 17 where the coating is in the form selected from the group consisting of a gel, membrane, foam, and fiber.

19. The method of claim 18 where the coating comprises a pharmacologically active agent.

20. A method of viscosupplementation for medical purposes which comprises contacting body tissue with a biocompatible viscoelastic gel slurry comprising a compound comprising hyaluronan cross-linked with a polyfunctional cross-linking agent having two or more aziridines, wherein the equivalent ratio of hyaluronan to aziridine is 1:1 to 1:10.

21. The method of claim 20 where the gel slurry further comprises a pharmacologically active agent.
